(19) United States
(12) Patent Application Publication
(10) Pub. No.: US 2026/0049162 A1
(43) Pub. Date: Feb. 19, 2026

(54) MULTIPLE-EFFECT MICROCIN CHITOSAN NANOPARTICLES, PREPARATION METHOD AND USE THEREOF

(71) Applicant: South China Agricultural University, Guangzhou (CN)

(72) Inventors: Weisheng CAO, Guangzhou (CN); Yu LI, Guangzhou (CN); Jinyu ZHANG, Guangzhou (CN)

(73) Assignee: South China Agricultural University, Guangzhou (CN)

(21) Appl. No.: 18/937,688

(22) Filed: Nov. 5, 2024

(30) Foreign Application Priority Data
Aug. 14, 2024 (CN) 202411115001.0

(51) Int. Cl. C08B 37/08; A01N 63/20; A01P 1/00; C07K 14/195

(52) U.S. Cl. CPC C08B 37/003; A01N 63/20; A01P 1/00; C07K 14/195

(57) ABSTRACT

Chitosan nanoparticles include chitosan and more than one lasso peptide, wherein the lasso peptide has a carboxyl group; an amino group in the structure of the chitosan is connected to the carboxyl group of the lasso peptide via an amide bond.

### CROSS-REFERENCE TO RELATED APPLICATION

This application claims the benefit of priority to Chinese Patent Application No. 202411115001.0, filed Aug. 14, 2024, the entire disclosure of which is incorporated herein by reference.

### Technical Field

The present disclosure belongs to the field of biomedicine and in particular relates to multiple-effect microcin chitosan nanoparticles, a preparation method and use thereof.

### BACKGROUND

The problem of drug resistance in Enterobacteriaceae caused by antibiotic abuse has attracted the attention of research in the international community. In order to reduce the dependence on antibiotics and ensure the health of livestock and poultry, antibiotic-free farming has gradually become the future development direction of animal husbandry in China. Previously, there was no ideal substitute product for antibiotics to deal with common Enterobacteriaceae in livestock and poultry breeding. However, microcin (Mcc) exhibits great potential in dealing with antibiotic resistance. Lasso peptides (LPs) are a class of ribosomally assembled and post-translationally modified small-molecule microcins (<10 kDa) with a “lasso” structure, which have stable and potent bactericidal biological activities. Common lasso peptides mainly include MccJ25, MccY, and Klebsidin, which can inhibit the activity of RNA polymerase after entering bacterial cells, thus resulting in a potent bactericidal effect (MIC=0.001-1.0 μg/mL). It has been found in previous studies that the new natural lasso peptide Microcin Y (MccY) has obvious complementary bactericidal advantages against Salmonella and Shigella as compared with MccJ25, whereas both MccJ25 and MccY exhibit insensitivity to Klebsiella, but the lasso peptide Klebsidin has a certain inhibitory effect on Klebsiella. Compared with other lasso peptides such as Citrocin, MccY, MccJ25 and Klebsidin have stronger bacteriostatic activities against Enterobacteriaceae and Gram-positive bacteria (MIC=0.001-20.0 μg/mL). Although lasso peptides have the potent bactericidal advantage, a single lasso peptide has targeting specificity for bacteria. Secondly, lasso peptide microcins commonly exhibit instability under acidic and basic conditions, and their sensitivity to digestive enzymes has also become the biggest obstacle in their use for feed.

### SUMMARY

The present disclosure aims at solving at least one of the above technical problems existing in the prior art. To this end, the present disclosure provides multiple-effect microcin chitosan nanoparticles comprising various lasso peptides coupled simultaneously, wherein the specific bacteriostatic effects of these different lasso peptides are coordinated to achieve a broad-spectrum antibacterial effect. In addition, such nanoparticles can improve the acid and base tolerance and safety of the lasso peptides, so as to break through the bottleneck in adding lasso peptides for use in livestock and poultry feed.

In order to achieve the above object of the present disclosure, a first aspect of the present disclosure provides chitosan (CN) nanoparticles comprising chitosan and more than one lasso peptide, wherein the lasso peptide has a carboxyl group; an amino group in the structure of the chitosan is connected to the carboxyl group of the lasso peptide via an amide bond.

Lasso peptides (LPs) are natural products of polypeptides found in bacteria, and they are a class of ribosomally synthesized and post-translationally modified bioactive peptides with lasso conformations. The structure of a lasso peptide usually contains 14-33 amino acid residues, wherein the 1st amino acid at the N-terminal forms a large amide ring with a side chain of the 7th, 8th, or 9th amino acid, and the amino acid at the C-terminal passes through this large amide ring to form a symbolic “lasso” structure. They can be divided into four classes according to the number of disulfide bonds contained in the lasso peptide and the location of formation thereof (as shown in FIG. 8). Lasso peptides in which the C-terminal peptide chain and the large peptide ring are connected via two disulfide bonds are defined as Class I lasso peptides (Class I). If, after the peptide chain tail exits the peptide ring, there is no disulfide bond linkage between the C-terminal peptide chain and the large peptide ring, but instead the lasso topology is stabilized through spatial interactions, such lasso peptides are classified as Class II lasso peptides (Class II). If, in lasso polypeptides, only one disulfide bond is formed between the sequence at the C-terminal end of the peptide chain tail and the peptide ring, such lasso peptides are classified as either Class III lasso peptides or Class IV lasso peptides (Class III or Class IV) based on the position of the disulfide bond. Specifically, in Class III lasso peptides, the disulfide bond connects the peptide ring to the peptide chain tail, whereas in Class IV lasso peptides, the disulfide bond only exists at the peptide chain tail. The four classes of lasso peptides as shown all have carboxyl groups.

In some embodiments of the present disclosure, the molar ratio of the chitosan to the lasso peptide is 5:(3-4).

In some embodiments of the present disclosure, the lasso peptide includes more than one of MccJ25, MccY, Klebsidin, Citrocin, Lihuanodin, Synthetase B1, Astexin 1, Koveensin, MccS, and MccC7.

In some embodiments of the present disclosure, the lasso peptide includes MccY and MccJ25.

In some embodiments of the present disclosure, the lasso peptide includes MccY and Klebsidin.

In some embodiments of the present disclosure, the lasso peptide includes MccJ25 and Klebsidin.

In some embodiments of the present disclosure, the lasso peptide includes MccY, MccJ25, and Klebsidin.

In the present disclosure, CN nanoparticles comprising a single lasso peptide MccY (CN-MccY) have no bacteriostatic effect on Serratia marcescens and Klebsiella pneumoniae; CN nanoparticles comprising a single lasso peptide MccJ25 (CN-MccJ25) have no bacteriostatic effect on Salmonella typhimurium; and CN nanoparticles comprising a single lasso peptide Klebsidin (CN-Klebsidin) have no bacteriostatic effect on Salmonella typhimurium.

Furthermore, in the present disclosure, the combined use of MccY and MccJ25 shows an additive bacteriostatic effect on Salmonella enteritidis, Salmonella typhimurium, Serratia marcescens, and Klebsiella pneumoniae and a synergistic bacteriostatic effect on Staphylococcus aureus; the combined use of MccY and Klebsidin shows an additive bacteriostatic effect on Salmonella enteritidis, Salmonella typhimurium, and Serratia marcescens and a synergistic bacteriostatic effect on Klebsiella pneumoniae and Staphylococcus aureus; and the combined use of MccJ25 and Klebsidin shows an additive bacteriostatic effect on Salmonella enteritidis, Klebsiella pneumoniae and Escherichia coli and a synergistic bacteriostatic effect on Serratia marcescens. Therefore, CN nanoparticles comprising MccY, MccJ25, and Klebsidin lasso peptides (CN-LPs) at least show a synergistic effect on Klebsiella pneumoniae, S. aureus, and Serratia marcescens and an additive bacteriostatic effect on Salmonella enteritidis and Salmonella typhimurium.

In the present disclosure, CN nanoparticles comprising MccY, MccJ25, and Klebsidin lasso peptides (CN-LPs) extend the bacteriostatic activity of microcins to the main food-borne pathogens of Salmonella, Serratia, and Klebsiella.

In some embodiments of the present disclosure, the molar ratio of CN to MccY to MccJ25 to Klebsidin is 5:(1-2):1:1.

In some embodiments of the present disclosure, the chitosan nanoparticles further comprise arginine (Arg), and an amino group in the structure of the chitosan is connected to a carboxyl group of arginine via an amide bond.

In some embodiments of the present disclosure, the molar ratio of CN to Arg is (1-10):1.

In the present disclosure, arginine is coupled with chitosan via an amide bond, thereby improving the stability of chitosan nanoparticles under acidic and basic conditions.

In some embodiments of the present disclosure, the particle size of the chitosan nanoparticles is 100 nm to 200 nm.

In a second aspect, the present disclosure provides a method for preparing chitosan nanoparticles, and the chitosan nanoparticles of the first aspect of the present disclosure are prepared by the method.

In some embodiments of the present disclosure, the preparation method comprises the following steps:
1) mixing a 1-ethyl-(3-dimethylaminopropyl) carbodiimide (EDC) solution with an N-hydroxysuccinimide solution (NHS) to obtain an EDCN mixed solution;
2) separately dissolving chitosan and a lasso peptide in acetylation solutions and mixing these acetylation solutions until uniform to obtain a chitosan acetylation solution and a lasso peptide acetylation solution;
3) adding the EDCN mixed solution in step 1) to the chitosan acetylation solution and lasso peptide acetylation solution in step 2), respectively, and mixing these acetylation solutions until uniform to obtain a chitosan-EDCN mixed solution and a lasso peptide-EDCN mixed solution; and
4) mixing the chitosan-EDCN mixed solution in step 3) with the lasso peptide-EDCN mixed solution in order and completing an esterification reaction under full stirring to prepare the chitosan nanoparticles.

In some embodiments of the present disclosure, step 2) further comprises the preparation of an arginine (Arg) acetylation solution, and the preparation step comprises dissolving arginine in an acetylation solution and mixing the mixture until uniform to obtain the arginine acetylation solution.

In some embodiments of the present disclosure, step 3) further comprises the preparation of an arginine-EDCN mixed solution, and the preparation step comprises adding the EDCN mixed solution in step 1) to the arginine acetylation solution in step 2) and mixing the mixture until uniform to obtain the arginine-EDCN mixed solution.

In some embodiments of the present disclosure, step 4) further comprises further mixing with the arginine-EDCN mixed solution in step 3).

In some embodiments of the present disclosure, the acetylation solution includes a sodium acetate solution, an acetyl chloride solution, an acetic anhydride solution, and a glacial acetic acid solution.

In some embodiments of the present disclosure, the acetylation solution includes a sodium acetate buffer (SAB).

In a third aspect, the present disclosure provides the use of the chitosan nanoparticles of the first aspect of the present disclosure in the preparation of a product with antibacterial effect.

In some embodiments of the present disclosure, the bacteria include Salmonella, Serratia, and Klebsiella.

In some embodiments of the present disclosure, the Salmonella includes Salmonella enteritidis and Salmonella typhimurium.

In some embodiments of the present disclosure, the Serratia includes Serratia marcescens.

In some embodiments of the present disclosure, the Klebsiella includes Klebsiella pneumoniae.

In some embodiments of the present disclosure, the product includes a feed, a preservative, a disinfectant, and a drug for external wound infection.

Beneficial effects of the disclosure include:
1) The chitosan nanoparticles containing chitosan and more than one lasso peptide as prepared by the present disclosure have a multiple-effect synergistic antibacterial effect on various bacteria, and the specific bacteriostatic effects of these different lasso peptides are coordinated to achieve a broad-spectrum antibacterial effect.
2) In the chitosan nanoparticles prepared by the present disclosure, there is also an arginine (Arg) linked via an amide bond, thereby improving the acid and base tolerance of the lasso peptides and breaking through the bottleneck in adding lasso peptides for use in livestock and poultry feed.
3) The chitosan nanoparticles prepared by the present disclosure have a relatively high safety and do not cause hemolysis even at a higher concentration.

### BRIEF DESCRIPTION OF DRAWINGS

FIG. 1 is a design diagram of the synthesis route of CN-LPs-Arg in Example 1.

FIG. 2A through FIG. 2D are the characterization analysis results of the nano-material CN-LPs-Arg in Example 2, wherein FIG. 2A is the FTIR analysis results of CN; FIG. 2B is the FTIR analysis results of CN-LPs-Arg; FIG. 2C is the particle size analysis results of CN-LPs-Arg; and FIG. 2D is the zeta potential characterization analysis diagram of CN-LPs-Arg.

FIG. 3A, FIG. 3B and FIG. 3C are transmission electron microscope images of three CN nanoparticles in Example 3: CN, CN-Arg, and CN-LPs-Arg, wherein FIG. 3A is transmission electron microscope image of CN nanoparticles in Example 3; FIG. 3B is transmission electron microscope image of CN-Arg nanoparticles in Example 3; FIG. 3C is transmission electron microscope image of CN-LPs-Arg nanoparticles in Example 3.

FIG. 4A through FIG. 4G are the physical and chemical stability test results of CN nanoparticles in Example 4, wherein FIG. 4A is the UPLC-Q-TOF mass spectrometry result of MccY; FIG. 4B is the UPLC-Q-TOF mass spectrometry result of Klebsidin; FIG. 4C is the UPLC-Q-TOF mass spectrometry result of MccJ25; FIG. 4D is the stability analysis results of MccY under the conditions of 95° C.; FIG. 4E is the stability analysis results of Mcc Y under the conditions of pH=2.0 and pH=12.0; FIG. 4F is the stability analysis results of CN-LPs-Arg under the conditions of 95° C., pH=2.0, and pH=12.0; and FIG. 4G is the antibacterial activity of CN-LPs-Arg after treatment under the conditions of 95° C., pH=2.0, and pH=12.0, as determined by a spot method.

FIG. 5 is the spot plate assay results of CN, Arg, and various CN nanoparticles (CN-Arg, CN-MccY, CN-MccJ25, CN-Klebsidin, CN-LPs, and CN-LPs-Arg), showing the bacteriostatic effect determined by the spot method; ** p<0.01, *** p<0.001, and **** p<0.0001.

FIG. 6 is the experimental process of combined bacteriostatic analysis (FICI) of the lasso peptides MccY, MccJ25, and Klebsidin.

FIG. 7A, FIG. 7B and FIG. 7C are the biocompatibility analysis results of CN nanoparticles, wherein FIG. 7A shows the morphology of red blood cells treated with CN nanoparticles; FIG. 7B is the cytotoxicity of CN nanoparticles to HEK293T cells as evaluated by an MTT method; and FIG. 7C shows the hemolysis of CN nanoparticles on red blood cells.

FIG. 8 is a structural schematic diagram of four classes of lasso peptides.

### DETAILED DESCRIPTION

The content of the present disclosure will be further explained in detail below by means of specific examples. Unless otherwise specially specified, the raw materials, reagents or devices used in the examples and comparative examples can all be obtained from conventional commercial channels or by methods in the prior art. Unless otherwise specially specified, experimental or test methods are all conventional methods in the art.

The instrument information used in the examples is as shown in Table 1 below:

TABLE 1
Instrument information used

| Instrument name | Company |
|---|---|
| ZHJH-C1214C ultra-clean workbench | Suzhou Purification Equipment Co., LTD |
| DK-8D electrothermal thermostatic water tank | Shanghai Yiheng Technology Co., LTD |
| GDS8000PC gel image formation analysis | UVP |
| system | |
| ND-1000 spectrophotometer | NanoDrop |
| T3000 Thermocycler PCR instrument | Whatman Biometra |
| Adjustable micropipette | Eppendorf, Germany |
| Power PacTM basic electrophoresis instrument | BIO-RAD |
| General centrifuge | Eppendorf, Germany |
| Chitosan CN | Sigma-Aldrich |
| N-(3-dimethylaminopropyl)-N- | Merck |
| ethylcarbodiimide hydrochloride (EDC) | |
| Thiazole blue tetrazole bromide | Merck |
| Penicillin-streptomycin | Gibco ™ThermoFisherTechnologies |
| DMEM | Gibco ™ThermoFisherTechnologies |
| Fetal Bovine Serum, FBS | Gibco ™ThermoFisherTechnologies |

GraphPad Prism 8.0 software is used for statistical analysis, and the intra-group statistical results are expressed as mean±SD. One-way ANOVA is used for comparison between two groups and three or more groups, respectively. Student's t test is used for significant difference comparison between groups, and each treatment is repeated three times independently.

### EXAMPLE 1: CHEMICAL SYNTHESIS OF CN-LPs-Arg

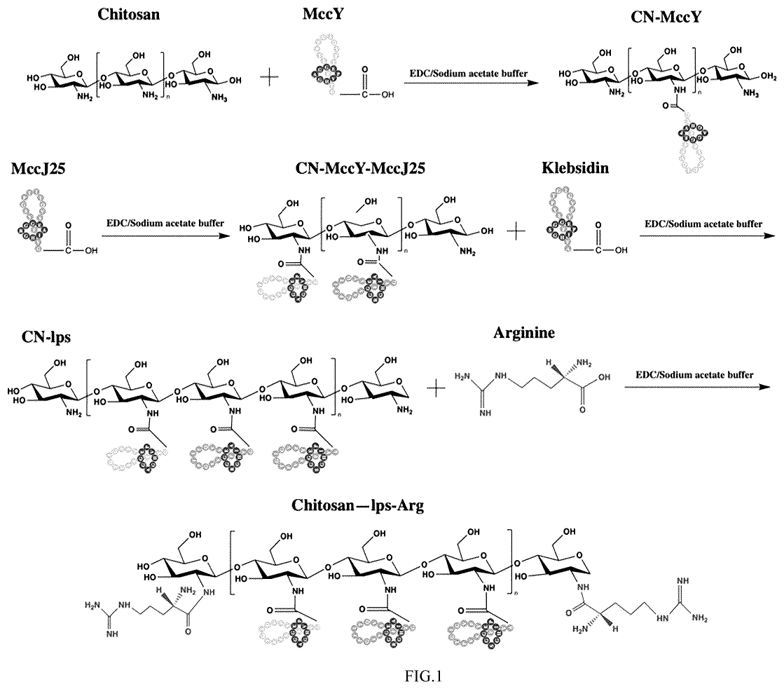

In this example, secreted proteins were obtained by an Escherichia coli prokaryotic expression system, and three lasso peptides (MccY, MccJ25, and Klebsidin) were then prepared by HPLC. For the preparation method, see reference 1. A multi-functional nano-material was obtained by coupling with chitosan (CN) by means of a chemical reaction. FIG. 1 depicts the synthesis process in detail, wherein by connecting an amino group of CN to a carboxyl group of a lasso peptide via an amide bond, various CN nanoparticles were synthesized, wherein the carboxyl groups of MccY, MccJ25, and Klebsidin were activated by EDC before successive coupling with CN to form CN-LPs. In addition, in order to improve the acid and base resistance, Arg was introduced to bind with CN-LPs to form CN-LPs-Arg. An amino group of CN-LPs and a carboxyl group of Arg were reacted in the presence of water-soluble EDC to form a new amide bond. The optimized formulation of these nano-materials achieved an encapsulation efficiency of more than 60% w/w in CN and encapsulation of about 90% of lasso peptides. The synthesis process of CN-LPs-Arg was specifically as follows:

The expression and preparation of the three lasso peptides were as follows: the vector pET-28a(+) was introduced into BL21 (DE3) competent cells to obtain BL21 (DE3) bacteria containing pET-28a(+), the BL21 (DE3) bacteria containing pET-28a(+) were revived in an LB plate with kanamycin at a concentration of 30 μg/mL, a loopful of bacteria was taken and inoculated into a 250 ml conical flask containing 100 mL of LB medium, kanamycin and isopropyl-β-D-thiogalactoside (IPTG) (the final concentration of kanamycin was 30 μg/mL and the final concentration of IPTG was 0.1 M) were added, and the conical flask was placed in a shaker for culturing (the culture conditions were 200 r/min, 37° C., and 14 h); and the bacterial liquid obtained after expression was collected and centrifuged at 5000 r/min for 20 min, the supernatant obtained after centrifugation was collected and filtered through a 0.22 μm filter membrane to obtain a supernatant filtrate, the supernatant was then subjected to protein enrichment by reverse solid phase extraction technology using an SPE C18 column, Q-TOF was then used to identify the expressed protein of interest, the protein of interest was then collected and purified by preparative HPLC and concentrated by reduced pressure distillation, and finally, the protein of interest was dried, weighed, and quantified by a freezing method.

The synthesis of CN-LPs-Arg was mainly divided into two steps. Step I. MccY, MccJ25, and Klebsidin were separately subjected to a coupling reaction with CN step by step. Preparation of reaction reagents: a 0.5 mM 1-ethyl-(3-dimethylaminopropyl) carbodiimide (EDC) solution and a 0.25 mM N-hydroxysuccinimide solution (NHS) were mixed at a volume ratio of 1:1 to form an EDCN solution. A 0.1 M sodium acetate buffer (SAB, pH=6.4) was prepared. CN was weighed and dissolved in SAB to prepare a 1 mg/mL CN acetylation solution. 100 μL of EDCN was added dropwise to activate the amino group of CN to produce a CN-EDCN solution, wherein the volume ratio of the EDCN solution to the CN acetylation solution was 1:10.1 mg of the three lasso peptides were separately dissolved in SAB to obtain 1 mg/mL lasso peptide SAB solutions. 100 μL of the EDCN solution was added and dissolved in the lasso peptide SAB solutions separately to activate the carboxyl groups of the lasso peptides to produce an MccY-EDCN solution, an MccJ25-EDCN solution, and a Klebsidin-EDCN solution, respectively, wherein the volume ratio of the EDCN solution to the lasso peptide SAB solution was 1:10.1 mL of the MccY-EDCN solution was added to 1 mL of the CN-EDCN solution, and completing an esterification reaction under full stirring to produce an EDCN solution of CN-MccY. 1 mL of the MccJ25-EDCN solution was added to 2 mL of the EDCN solution of CN-MccY and stirred for an esterification reaction to produce an EDCN solution of CN-MccY-MccJ25, and 1 mL of the Klebsidin-EDCN solution was then added to 3 mL of the EDCN solution of CN-MccY-MccJ25 and fully stirred to complete an esterification reaction to produce an EDCN solution of CN-LPs.

Step II. Arg was coupled with an amino group of CN under certain conditions to form a new amide bond, thereby completing amino acid modification on CN-LPs to synthesize CN-LPs-Arg, wherein the introduction of Arg could further improve the acid- and base-resistant stability of CN-LPs. Specifically, 1 mg of Arg was dissolved in 1 mL of an SAB solution to prepare a 1 mg/mL Arg SAB solution, and the EDCN solution prepared in Step I (100 μL) was added dropwise to activate the carboxyl group of Arg to produce an Arg-EDCN solution, wherein the volume ratio of the EDCN solution to the Arg SAB solution was 1:10.1 mL of the Arg-EDCN solution was added to 1 mL of the EDCN solution of CN-LPs, distilled water was charged using an MWCO 2000 benzoylation dialysis tube, and dialysis was carried out continuously 3 times for 24 h to remove excessive reactants and by-products. Finally, CN-LPs-Arg was obtained, and the obtained solution was freeze-dried for use.

The preparation methods for CN-MccY, CN-Arg, CN-MccJ25, and CN-Klebsidin as control samples were similar to the above preparation method for CNLPs-Arg. EDCN solutions of CN-MccY, CN-Arg, CN-MccJ25, and CN-Klebsidin were prepared by activation of EDCN and esterification with the CN-EDCN solution in step I, and after dialysis and freeze drying in step II, CN-MccY, CN-Arg, CN-MccJ25, and CN-Klebsidin were then obtained.

### EXAMPLE 2: CHARACTERIZATION ANALYSIS OF CN-LPs-Arg

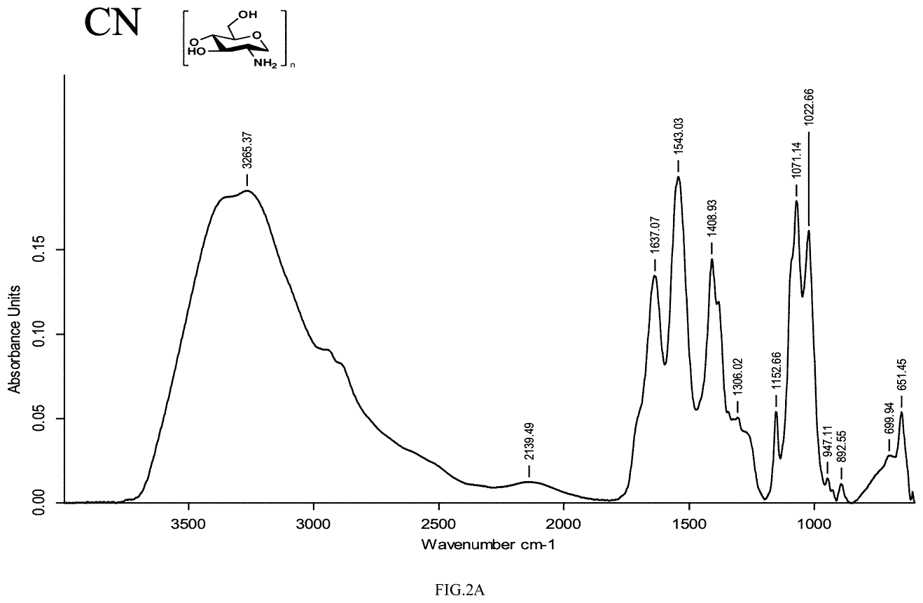

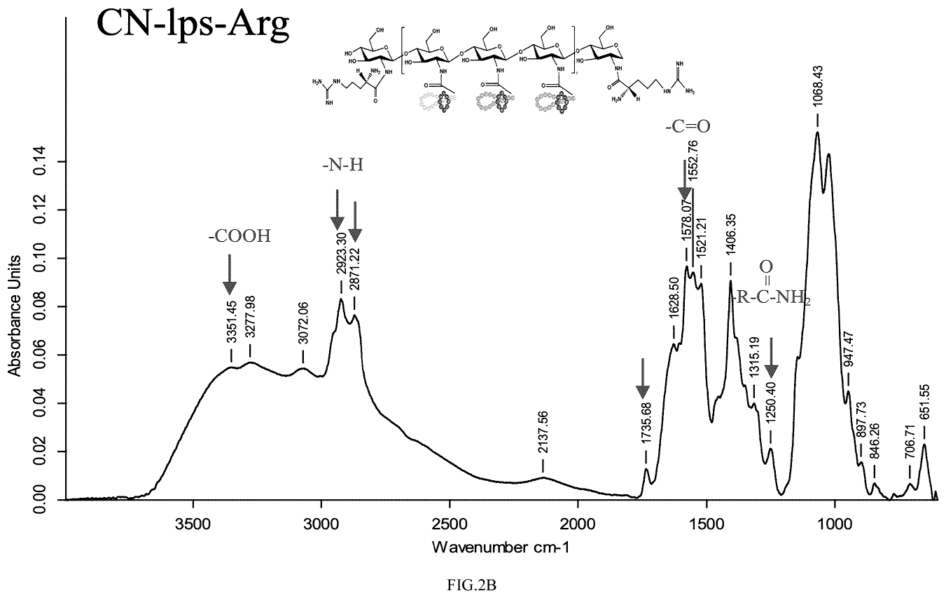

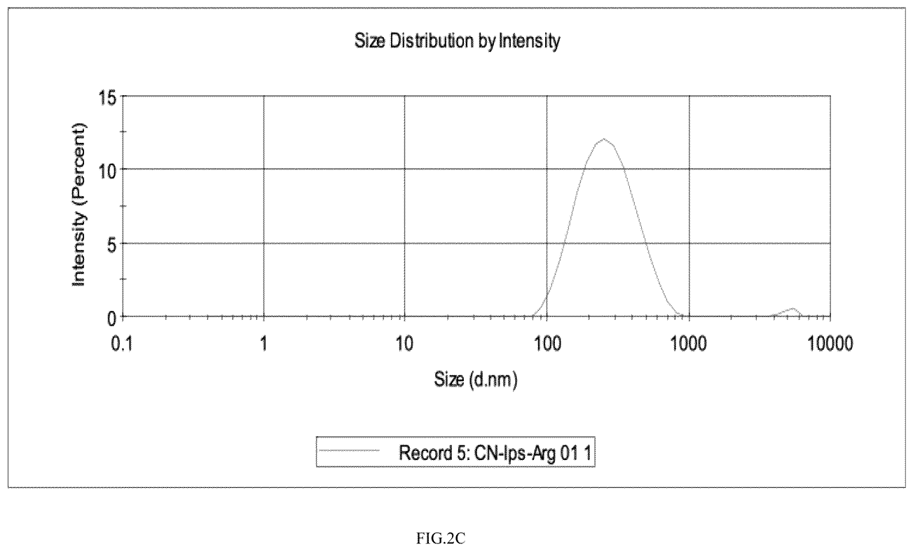

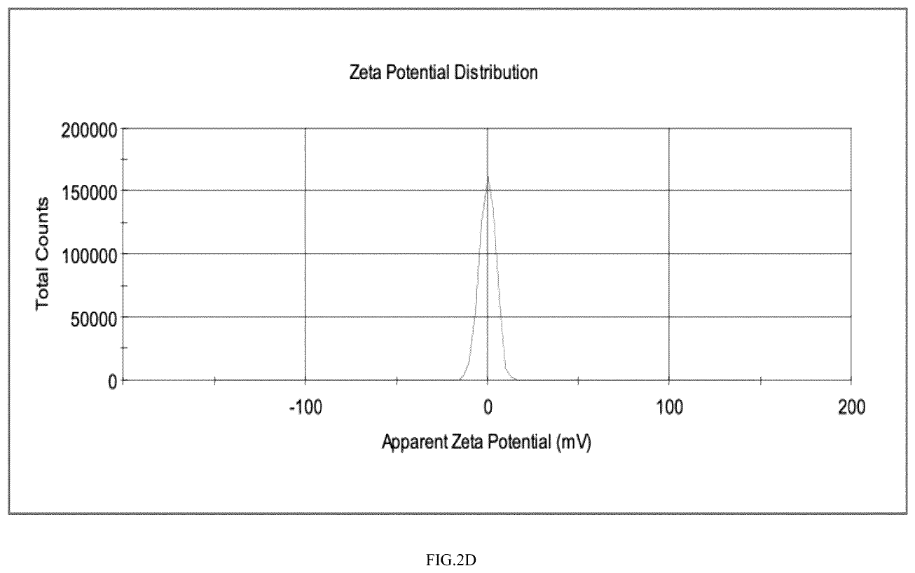

Vertex 70 infrared spectrometer (Fourier Transform Infrared Spectrometer, FTIR) was used to study the conjugation efficiency of CN-LPs-Arg, CN-Arg, and CN. The spectral range was between 4000-15000 cm⁻¹, the resolution was 4 cm⁻¹, and the number of scanning was 64. The structures of CN and CN-LPs-Arg were tested spectroscopically by FT-IR, and the results were as shown in FIG. 2A and FIG. 2B. CN had characteristic peaks of N—H at around 2850 and 2930 cm⁻¹, CN-LPs had N—H stretching bands at 2921.99 and 2858.79 cm⁻¹, and CN-LPs-Arg had N—H stretching bands at 2923.30-2871.22 cm⁻¹. A stretching band of —COOH was observed at about 3359.96 cm⁻¹ for CN-LPs and 3351.45 cm⁻¹ for CN-LPs-Arg, respectively. A new peak related to the reaction of the CN structure and carboxyl appeared at 1735 cm⁻¹, which was represented by the characteristic peak of —C—O, confirming a successful reaction between the amino group of CN and the carboxyl group of the lasso peptide. The infrared absorption peak of —C═O generally appears in a range of 1700-1750 cm⁻¹; however, since CN has a high degree of deacetylation, the infrared absorption peak thereof is relatively weak. Due to the occurrence of the reaction, the vibration peak of the amide group in CN-LPs-Arg was obvious. The particle size and zeta potential of the synthesized compound were determined by Malvern ZS-90 Zetasizer at 25° C. After modification, the static number average particle size of CN-LPs-Arg increased slightly, ranging from 200 nm to 400 nm, as shown in FIG. 2C. The lower molecular weight of CN-LPs-Arg resulted in a more uniform pore morphology and a lower pore polydispersity. The zeta potential value of the prepared nano-material was between-22.0 and 22.0 mV, as shown in FIG. 2D.

### EXAMPLE 3: TRANSMISSION ELECTRON MICROSCOPE ANALYSIS OF CN-LPs-Arg

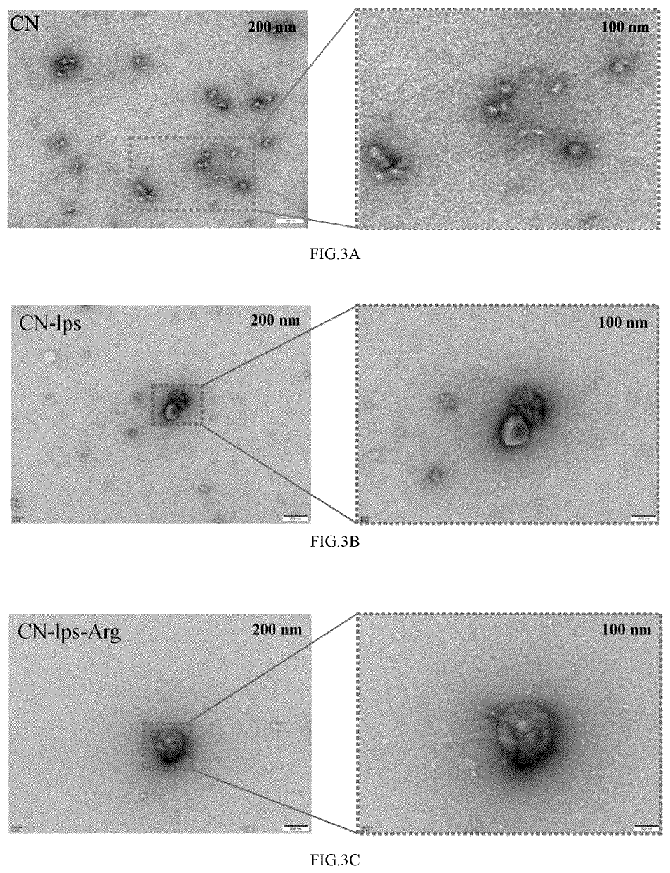

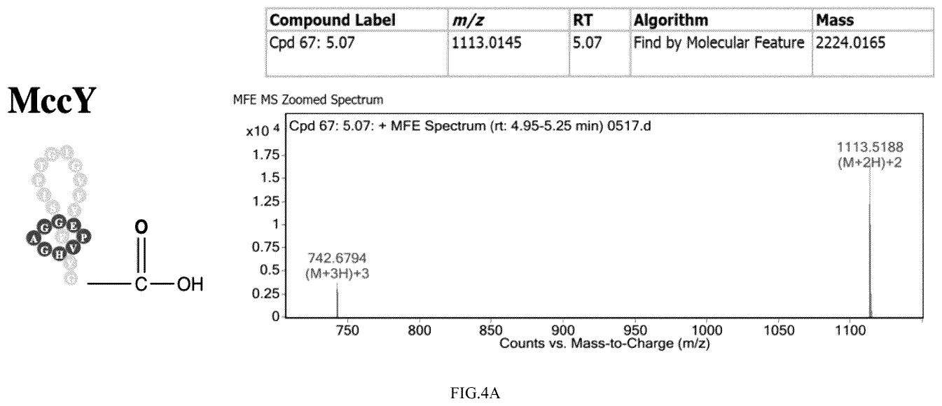

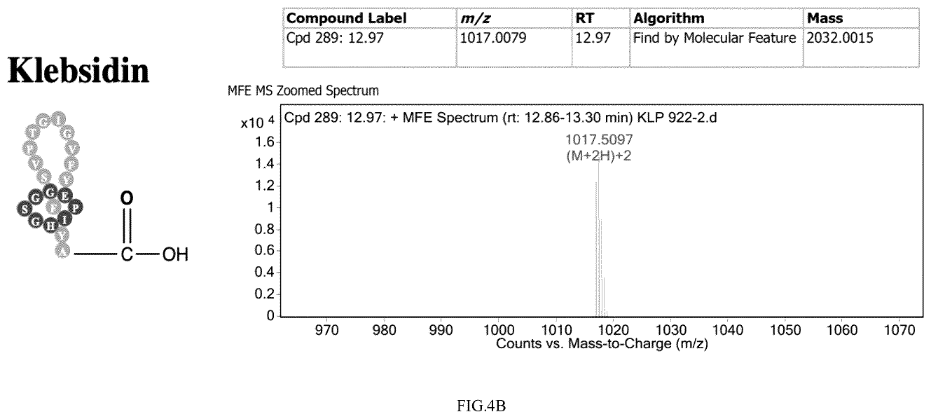

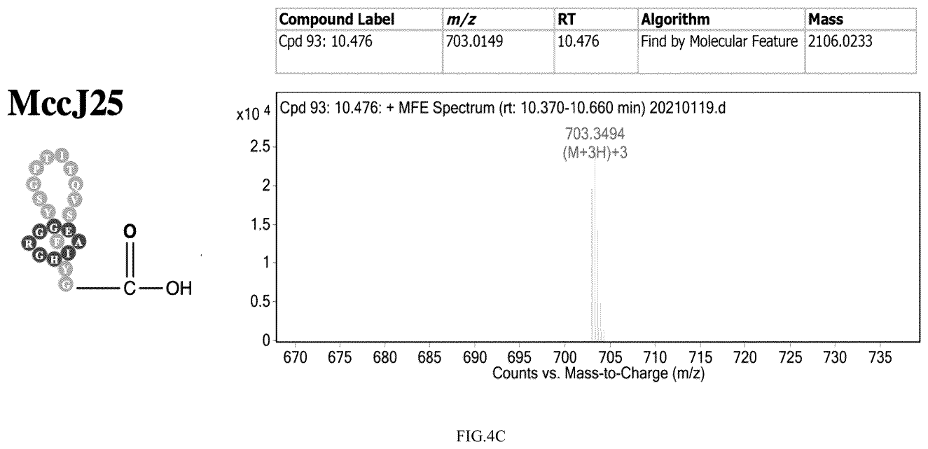

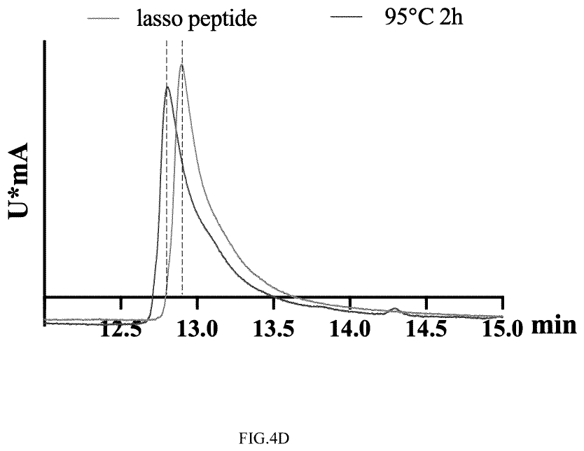

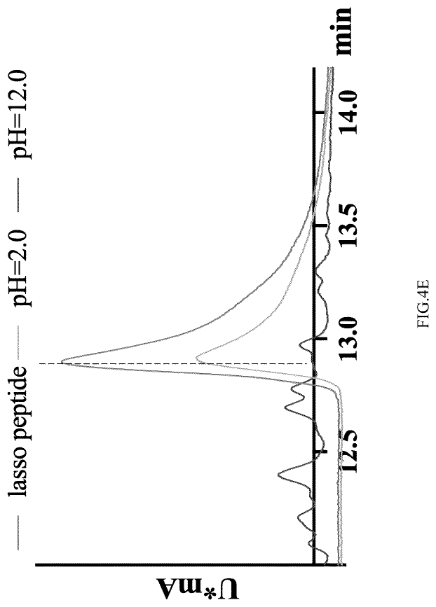

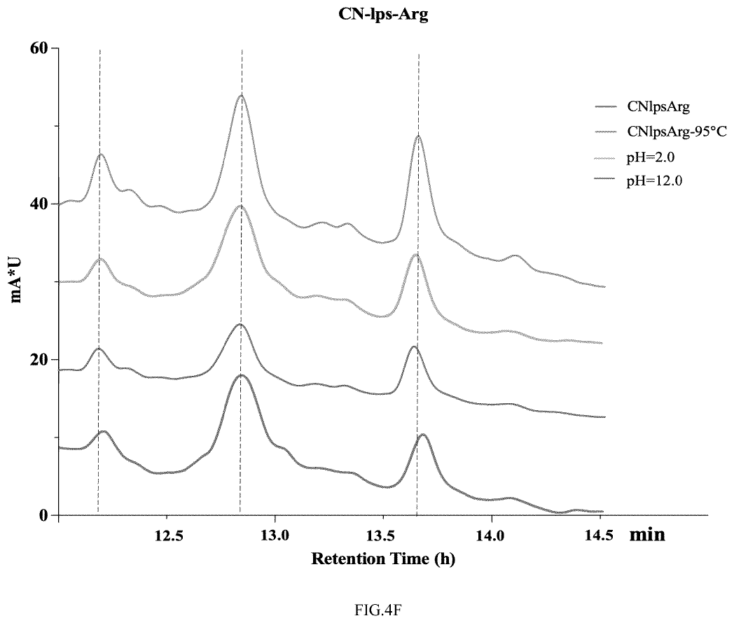

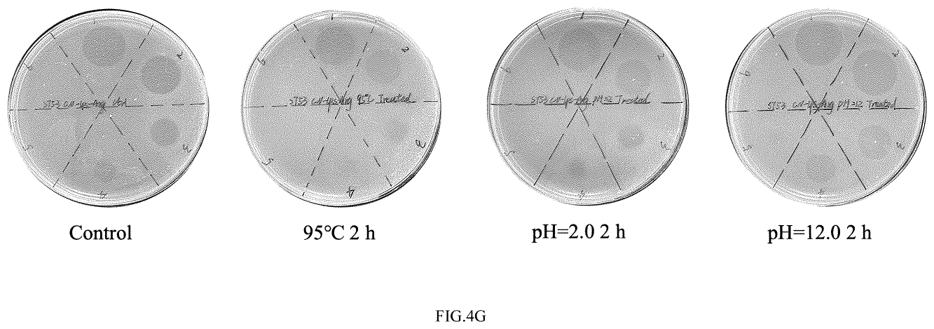

CN-LPs-Arg nanoparticles were resuspended in a 0.1M sodium acetate solution to obtain a 1 mg/mL suspension of CN-LPs-Arg nanoparticles, 20 μL of the suspension was dropwise added to a 200-mesh grid, incubated for 10 min at room temperature, and then negatively stained with 2% phosphotungstic acid for 3 min, and the remaining liquid was filtered out with filter paper. The samples were observed by HT7700 and Hitachi Su-8010 transmission electron microscopes. The structures and particle sizes of the three nanoparticles, CN, CN-Arg, and CN-LPs-Arg, were as shown in FIG. 3A, FIG. 3B and FIG. 3C. In FIG. 3A, the particle size of CN was between 10 nm and 50 nm; and in FIG. 4B and FIG. 4C, the particle sizes of CN-LPs and CN-LPs-Arg were separately between 100 nm and 200 nm.

### EXAMPLE 4: PHYSICAL AND CHEMICAL STABILITY TEST OF CN-LPs-Arg

In order to investigate the stability of chitosan nanoparticles, an acid-base extreme test and a high-temperature stability test were carried out.

Firstly, the molecular weights of the three lasso peptides were analyzed by UPLC-Q-TOF mass spectrometry. The three lasso peptides were expressed and prepared according to Example 1. The UPLC-Q-TOF mass spectrometry analysis results showed that the exact molecular weight of MccY was 2224.0165 m/z, and the corresponding ionization molecular weights of (M+3H)⁺³ and (M+2H)⁺² were respectively 742.6794 m/z and 1113.5188 m/z (FIG. 4A). Klebsidin consisting of only 19 amino acids had a molecular weight of 2032.0015 m/z, and the corresponding ionization molecular weight of (M+2H)⁺² was 1017.5097 m/z (FIG. 4B). As can be seen from FIG. 4C, the exact molecular weight of MccJ25 was 2106.0223 m/z, and the corresponding ionization molecular weight of (M+3H)⁺³ was 703.3494 m/z.

The experimental steps of the high-temperature stability test involved dissolving MccY in a 0.1 M SAB solution to prepare a 1 mg/mL MccY SAB solution, and then dividing MccY into two groups: an experimental group which was treated at 95° C. for 2 h and a control group which was placed at room temperature for 2 h.

The experimental steps of the acid-base extreme test involved dividing the 1 mg/mL MccY SAB solution into three groups: a first group which was treated in a hydrochloric acid solution with pH=2.0 for 2 h, a second group which was treated in a sodium hydroxide solution with pH=12.0 for 2 h, and a third group which was a control group without any treatment.

Ultraviolet scanning spectroscopy at 280 nm was then used to detect the lasso peptide MccY. The results were as shown in FIG. 4D and FIG. 4E, in which the degradation of the lasso peptide MccY was mainly due to the acid and base treatments with pH=2.0 and pH=12.0, and the high-temperature treatment had no effect on the MccY protein. The acid treatment weakened the characteristic absorption peak of MccY. That is, the acid treatment resulted in partial peptide degradation. In addition, additional degradation peaks were observed in the spectrum at 12.7-13.3 min after the base treatment.

The CN-LPs-Arg nanoparticles were further subjected to a stability test by means of a high-temperature stability test and an acid-base extreme test. The CN-LPs-Arg nanoparticles were dissolved in a 0.1 M SAB solution to prepare a 1 mg/mL CN-LPs-Arg SAB solution. The other experimental steps were as above. The CN-LPs-Arg nanoparticles were then detected by ultraviolet scanning spectroscopy at 280 nm. The results were as shown in FIG. 4F, wherein the CN-LPs-Arg nanoparticles in the acid and base treatments, high-temperature treatment, and control groups all showed characteristic peaks at 12.2 min, 12.7 min, and 13.6 min, respectively. The above results showed that the CN-LPs-Arg nanoparticles can improve the stability of lasso peptides in acidic and basic and high-temperature environments, and the CN-LPs-Arg nanoparticles are stable in the acidic and basic and high-temperature environments and have acid and base resistance and high-temperature resistance.

After 0.1 mg/mL CN-LPs-Arg was treated for 2 h at 95° C., pH=2.0 and pH=12.0, respectively, the bactericidal activity thereof was determined by a spot method. 10 μL of bacteria to be tested with OD₆₀₀=0.8 (Salmonella typhimurium, ST53, the information was as shown in Table 2) was taken and cultured in an LB solid culture medium, and the treated lasso peptide was then diluted into six concentrations and spotted to six fan-shaped regions, wherein the drug concentrations from the 1st well to the 6th well were: region 1:10.0 μM, region 2:5.0 μm, region 3:1.0 μm, region 4:0.5 μm, region 5:0.2 μM, and region 6:0.04 μM. They were cultured at 37° C. for 12 h. The results were as shown in FIG. 4G. The high-temperature treatment resulted in a 5-fold decrease in activity (0.2 μM), whereas in the two groups undergoing the acid and base treatments, the activity was equivalent to that of the control group (0.04 μM). It can be seen that the chitosan arginine nanoparticles CN-LPs-Arg had the stability and acid and base buffering effect of nanoparticles, and the acid and base resistance of the three lasso peptides in CN-LPs-Arg was improved.

### EXAMPLE 5: BACTERIOSTATIC ACTIVITY OF CN-LPs-Arg

The synthesized samples (CN, Arg, CN-Arg, CN-MccY, CN-MccJ25, CN-Klebsidin, CN-LPs, and CN-LPs-Arg) were subjected to a spot plate assay to exhibit the antibacterial effect determined by a spot method. The strains to be tested included Salmonella enteritidis (SE63), Salmonella typhimurium (ST53), Serratia marcescens (SMATCC14756), and Klebsiella pneumoniae (KP07). The specific information about the strains was as shown in Table 2. The specific steps comprised culturing the strain on an LB agar plate overnight, then transferring the strain to an LB broth, and culturing the strain at 37° C. until the OD₆₀₀ value reached 0.8. Subsequently, the bacterial liquid was diluted at a ratio of 1:1000 and added to an LB solid medium containing 0.5% agar, which was then prepared into a plate at 40° C. Then, each LB plate was divided into eight fan-shaped regions. From the 1st to the 8th regions, the plate was spotted and inoculated with the following drugs at 0.05 μM in a dosage amount of 10-20 μL:1. CN, 2. Arg, 3. CN-Arg, 4. CN-MccY, 5. CN-MccJ25, 6. CN-Klebsidin, 7. CN-LPs, and 8. CN-LPs-Arg, respectively. After 24 hours of culture, the bacteriostatic diameter was observed and recorded. In the spot plate assay, the inhibition zone determination results of CN nanoparticles against each strain were as shown in Table 3.

TABLE 2
Strains used in Example 5

| Strain | Related information | Source |
|---|---|---|
| SE63 | Salmonella enteritidis, | Reference 1 |
| | wide type strain | |
| ST53 | Salmonella typhimurium, | Reference 1 |
| | wide type strain | |
| SMATCC14756 | Serratia marcescens, ATCC strain | ATCC |
| KP07 | Klebsiella pneumoniae, ATCC strain | ATCC |

TABLE 3
Inhibition zone determination of CN nanoparticles in spot plate assay

| | | Diameter of | | | Diameter of |
|---|---|---|---|---|---|
| | Treatment | inhibition | | Treatment | inhibition |
| Strain | group | zone (cm) | Strain | group | zone (cm) |
| SE63 | CN | 0.00 ± 0.00 | | CN | 0.00 ± 0.00 |
| | Arg | 0.00 ± 0.00 | | Arg | 0.00 ± 0.00 |
| | CN-Arg | 0.96 ± 0.04 | | CN-Arg | 0.99 ± 0.03 |
| | CN-MccY | 1.44 ± 0.01 | SMATCC | CN-MccY | 0.00 ± 0.00 |
| | CN-MccJ25 | 2.71 ± 0.03 | 14756 | CN-MccJ25 | 2.50 ± 0.01 |
| | CN-Klebsidin | 2.91 ± 0.01 | | CN-Klebsidin | 2.90 ± 0.02 |
| | CN-LPs | 2.30 ± 0.01 | | CN-LPs | 2.25 ± 0.13 |
| | CN-LPs-Arg | 2.49 ± 0.01 | | CN-LPs-Arg | 2.44 ± 0.08 |
| ST53 | CN | 0.00 ± 0.00 | | CN | 0.00 ± 0.00 |
| | Arg | 0.00 ± 0.00 | | Arg | 0.00 ± 0.00 |
| | CN-Arg | 0.98 ± 0.03 | | CN-Arg | 1.50 ± 0.04 |
| | CN-MccY | 2.64 ± 0.03 | KP07 | CN-Mcc Y | 0.00 ± 0.00 |
| | CN-MccJ25 | 0.00 ± 0.00 | | CN-MccJ25 | 2.21 ± 0.03 |
| | CN-Klebsidin | 0.00 ± 0.00 | | CN-Klebsidin | 2.70 ± 0.04 |
| | CN-LPs | 1.71 ± 0.02 | | CN-LPs | 1.68 ± 0.02 |
| | CN-LPs-Arg | 2.13 ± 0.05 | | CN-LPs-Arg | 2.18 ± 0.17 |

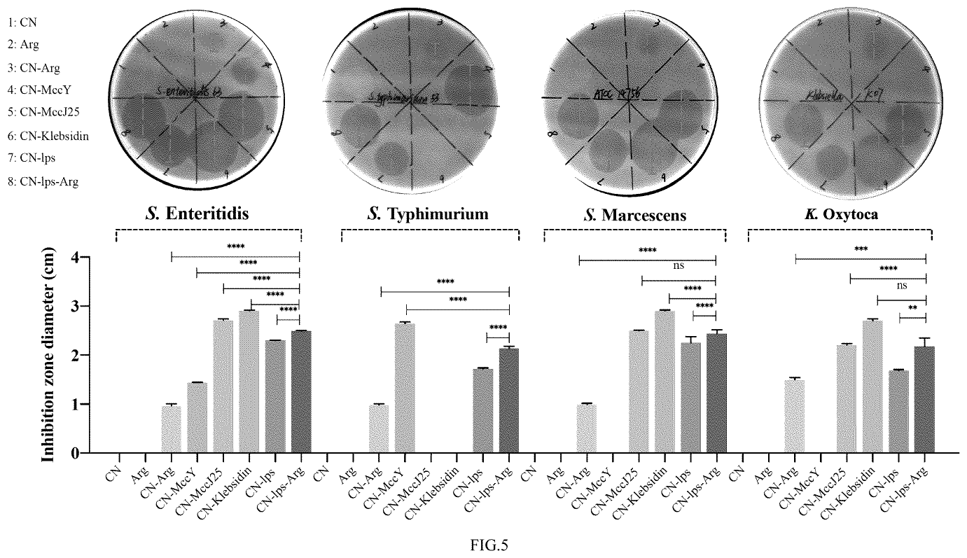

Furthermore, the statistical results of the spot plate assay were as shown in FIG. 5, in which CN and Arg alone had no inhibitory effect on these microorganisms, and CN nanoparticles with individual lasso peptides (CN-MccY, CN-MccJ25, and CN-Klebsidin) had respectively specific inhibitory effects on specific strain types, whereas CN nanoparticles with the three lasso peptides (CN-LPs) had a bacteriostatic effect on all of Salmonella enteritidis (SE63), Salmonella typhimurium (ST53), Serratia marcescens (SMATCC14756), and Klebsiella pneumoniae (KP07). That is to say, CN-LPs achieved a multiple-effect synergistic bacteriostatic effect on various bacteria. Compared with the CN nanoparticles with individual lasso peptides (CN-MccY, CN-MccJ25, and CN-Klebsidin), the concentrations of the corresponding individual lasso peptides in the CN-LPs at the same concentration were reduced to about ⅓ of those in the CN nanoparticles with these individual lasso peptides, so the diameter of inhibition zone for some strains was smaller. In addition, compared with CN-LPs, further coupling of CN nanoparticles with Arg (CN-LPs-Arg) can further improve the bacteriostatic effect on various bacteria.

### EXAMPLE 6: COMBINED BACTERIOSTATIC ANALYSIS (FICI) of MccY, MccJ25, AND KLEBSIDIN

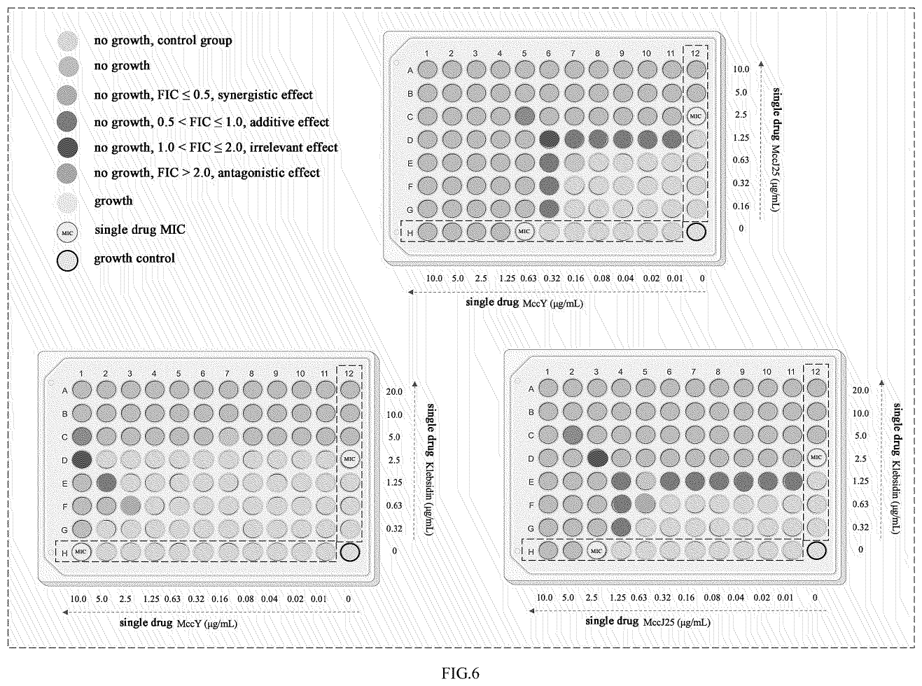

In order to confirm the bacteriostatic relationship in drug combinations between the three individual lasso peptides, and the relationships between MccY and MccJ25, MccY and Klebsidin, and MccJ25 and Klebsidin were further explored. The experimental process was as shown in FIG. 6, and the experimental results were as shown in Table 4-Table 6. FICI experiment: In each well of a 96-well plate, 50 μL of lasso peptide A at a specific concentration and 50 μL of lasso peptide B at a specific concentration were added in equal volumes. The concentrations of the lasso peptides were as shown in FIG. 6. The well plate was placed in a 37° C. environment and incubated for 24 h to observe and record the MIC of each lasso peptide. Each experimental concentration was repeated three times to ensure the accuracy of the results. According to the calculation formula of FICI (combined bacterial inhibition fraction), the interaction between lasso peptide A and lasso peptide B was evaluated. The calculation formula of FICI (combined bacterial inhibition) was as follows:

FICI=MIC (lasso peptide A in combined use)/MIC (lasso peptide B used alone)+MIC (lasso peptide B in combined use)/MIC (lasso peptide A used alone)

FICI>2 indicated an antagonistic effect between lasso peptides A and B; 1<FICI≤2 indicated an irrelevant effect; 0.5<FICI≤1 indicated an additive effect between lasso peptides A and B, and FICI≤0.5 indicated a synergistic effect between lasso peptides A and B.

TABLE 4
FICI numerical value results of MccY and MccJ25

| | MIC of | MIC of | MIC | | |
|---|---|---|---|---|---|
| | MccY | MccJ25 | (MccY/ | | |
| Strain | (μg/mL) | (μg/mL) | MccJ25) | FICI | Effect |
| S. enteritidis | 5 | 0.16 | 2.5/0.08 | 0.75 | Additive |
| S. typhimurium | 0.5 | 0.5 | 0.13/0.31 | 0.88 | Additive |
| S. marcescens | 5 | 0.16 | 0.08/0.08 | 0.516 | Additive |
| K. oxytoca | 0.63 | 2.5 | 0.16/0.63 | 0.506 | Additive |
| E. coli | 0.31 | 0.16 | 0.16/0.16 | 1.50 | Irrelevant |
| S. aureus | 1.25 | 2.5 | 0.31/0.31 | 0.372 | Synergistic |

TABLE 5
FICI numerical value results of MccY and Klebsidin

| | MIC of | MIC of | MIC | | |
|---|---|---|---|---|---|
| | MccY | MccJ25 | (MccY/ | | |
| Strain | (μg/mL) | (μg/mL) | MccJ25) | FICI | Effect |
| S. enteritidis | 0.5 | 20 | 0.13/10 | 0.51 | Additive |
| S. typhimurium | 0.32 | 10 | 0.16/1.25 | 0.625 | Additive |
| S. marcescens | 5 | 2.5 | 0.16/1.25 | 0.532 | Additive |
| K. oxytoca | 5 | 20 | 0.16/5 | 0.282 | Synergistic |
| E. coli | 0.16 | 20 | 0.16/0.63 | 1.03 | Irrelevant |
| S. aureus | 5 | 2.5 | 0.63/0.31 | 0.25 | Synergistic |

TABLE 6
FICI numerical value results of MccJ25 and Klebsidin

| | MIC of | MIC of | MIC | | |
|---|---|---|---|---|---|
| | MccY | MccJ25 | (MccY/ | | |
| Strain | (μg/mL) | (μg/mL) | MccJ25) | FICI | Effect |
| S. enteritidis | 0.16 | 5 | 0.08/0.31 | 0.562 | Additive |
| S. typhimurium | 0.63 | 10 | 0.63/2.5 | 1.25 | Irrelevant |
| S. marcescens | 2.5 | 2.5 | 0.16/0.16 | 0.128 | Synergistic |
| K. oxytoca | 2.5 | 2.5 | 0.63/1.25 | 0.752 | Additive |
| E. coli | 0.16 | 20 | 0.08/5 | 0.75 | Additive |
| S. aureus | 0.63 | 2.5 | 0.63/1.25 | 1.50 | Irrelevant |

The results in Table 4, Table 5, and Table 6 showed that (1) during drug testing, MccY and MccJ25 exhibited an additive effect on Salmonella enteritidis (SE63), Salmonella typhimurium (ST53), Serratia marcescens (SMATCC14756), and Klebsiella (KP07); (2) During drug testing, MccY and Klebsidin exhibited an additive effect on Salmonella enteritidis (SE63), Salmonella typhimurium (ST53), and Serratia marcescens (SMATCC14756) and a synergistic effect on Klebsiella (KP07); (3) During drug testing, MccJ25 and Klebsidin exhibited an additive effect on Salmonella enteritidis (SE63) and Klebsiella (KP07), an irrelevant effect on Salmonella typhimurium (ST53), and a synergistic effect on Serratia marcescens (SMATCC14756).

### EXAMPLE 7: BIOCOMPATIBILITY ANALYSIS OF CN1.25-LPs-Arg

The effect of CN-LPs-Arg on the activity of mammalian cells was evaluated for its biocompatibility. The specific steps involved collecting human red blood cells in a collection tube containing heparin. The steps were as follows: centrifugation at 750 g for 15 min to separate serum, washing the red blood cells three times with sterile PBS, and then diluting the red blood cells with PBS to prepare a 1% red blood cell suspension; and then treating the cells with samples (CN-Arg, CN-LPs, and CN-LPs-Arg) with a concentration of 10 μg/mL and observing their morphological changes. Triton X-100 diluted with PBS was used as a positive control, and PBS-treated cells were used as a negative control.

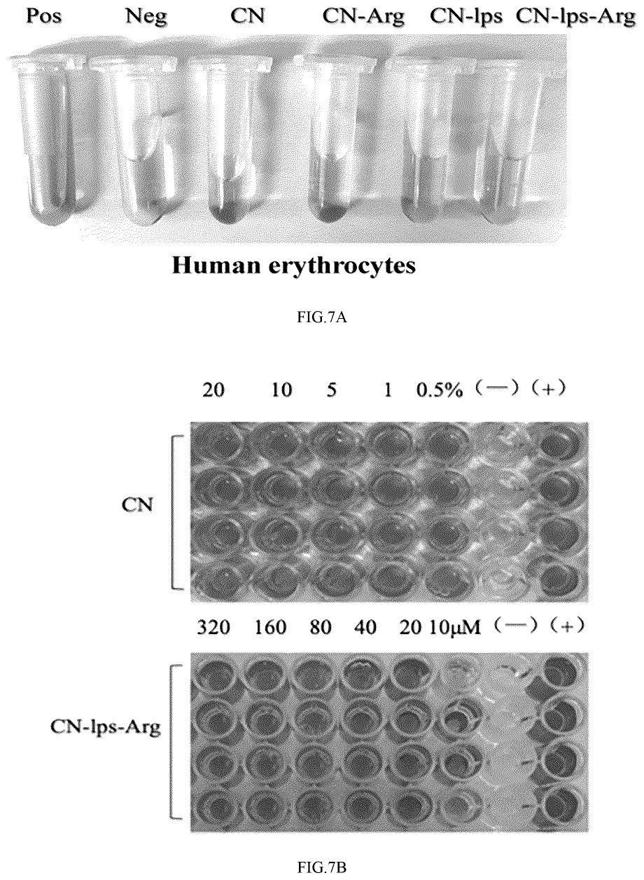

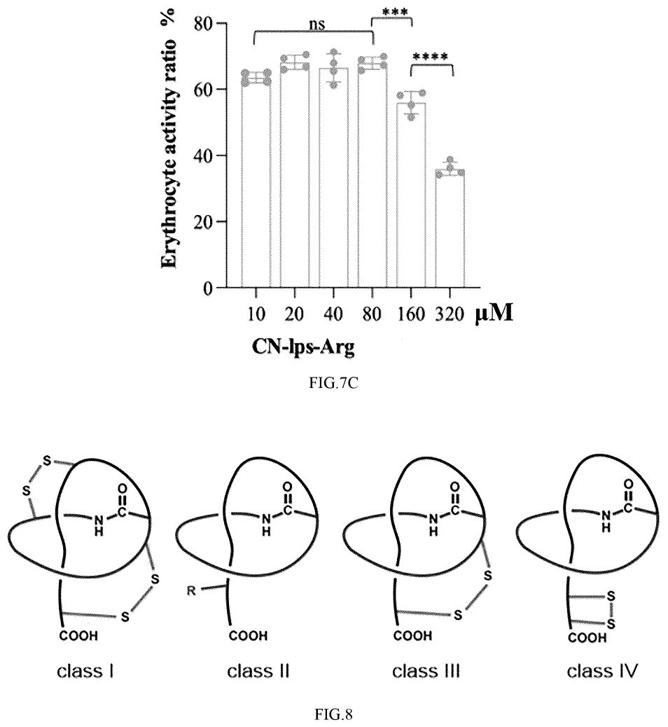

The experimental results were as shown in FIG. 7A, in which the negative control cells had normal morphology, whereas the positive control cells treated with Triton X-100 showed cell membrane lysis and cell death, and none of the cells treated with CN, CN-Arg, CN-LPs, and CN-LPs-Arg caused obvious cell membrane lysis and hemolysis.

Next, the cytotoxicity of CN nanoparticles to HEK293T cells was evaluated by an MTT method. 293T cells were cultured at 37° C. and 5% CO₂. The 293T cells were cultured in DMEM to which 10% fetal bovine serum and 1% penicillin/streptomycin were added. 200 μL of the above 1% red blood cell suspension was taken (inoculated into a 96-well plate at a concentration adjusted to 1.0×10⁵ cells/well) and cultured for 24 h. CN nanoparticles (CN-LPs-Arg and CN) were then resuspended and diluted in DMEM. Based on a concentration of 20.0% (20 mg/mL), the CN nanoparticles were diluted in a gradient manner to concentrations of 0.5%, 1.0%, 5.0%, 10.0%, and 20.0%; and the drug CN-LPs-Arg was diluted in a gradient manner to concentrations of 10 μM, 20 μM, 40 μM, 80 μM, 160 μM, and 320 μM. Then, 200 μL of the diluted CN nanoparticle solution was added to each well and incubated for 24 h at 37° C. and 5% CO₂. Triton X-100 diluted with PBS was used as a positive control, and PBS-treated cells were used as a negative control.

The qualitative results of the cytotoxicity of CN nanoparticles to HEK293T cells were as shown in FIG. 7B. FIG. 7B reflected the color change of the red blood cells at different concentrations. The smaller the red color change, the lower the degree of hemolysis. The red blood cells within the concentration ranges of 0.5%-20.0% and 10 μM-80 μM had no obvious color change, whereas the color change was relatively great at concentrations of 160 μM and 320 μM, indicating erythrolysis.

The 1% red blood cell suspension prepared above was used. In the experimental group, 100 μL of the red blood cell suspension and 100 μL of CN-LPs-Arg samples (concentrations: 10 μM, 20 μM, 40 μM, 80 M, 160 μM, and 320 μM) were separately added to microcentrifuge tubes. The positive control was 100 μL of a red blood cell suspension treated with a diluted solution of Triton X-100 in sterile PBS (final concentration=0.5 mg/mL), and the negative control was a red blood cell suspension treated with PBS. The control group and the experimental group were incubated at 37° C. for 2 h. After centrifugation, 50 μL of the supernatant was taken and transferred to a 96-well plate. The absorbance of the supernatant at 540 nm in the control group and the experimental group was determined by spectrophotometry, and the hemolysis rate was determined. The calculation formula of hemolysis rate in percentage was as follows:

$$Hemolysis rate \% = ( {Abs}_{Compound} - {Abs}_{Positive} ) / ( {Abs}_{Negative} - {Abs}_{Blank} ) \times 100 \%$$

wherein Abs_{Compound} represented the absorbance in the experimental group treated with the CN nanoparticle sample to be tested, Abs_{Positive} represented the absorbance of the positive control, Abs_{Negative} represented the absorbance of the negative control, and Abs_{Blank} represented the absorbance of the blank control.

The hemolysis rate results were as shown in FIG. 7C. CN-LPs-Arg had no effect on the activity of red blood cells at a concentration below 80 μM. Compared with Triton X-100-induced complete hemolysis of red blood cells, the CN nanoparticles had no hemolytic toxicity. This indicated that the CN-LPs-Arg nanoparticles were not enough to cause obvious side effects in the host.

The above examples are preferred embodiments of the present disclosure, and the embodiments of the present disclosure are not limited by the above examples. Any other changes, modifications, replacements, combinations, and simplifications made without departing from the spirit and principle of the present disclosure shall be equivalent substitutions and are all included in the scope of protection of the present disclosure.

### REFERENCE

1. Li, Y., Han, Y., Zeng, Z., Li, W., Feng, S., & Cao, W. (2021). Discovery and Bioactivity of the Novel Lasso Peptide Microcin Y. Journal of agricultural and food chemistry, 69 (31), 8758-8767.

1. Chitosan nanoparticles comprising chitosan and more than one lasso peptide, wherein the lasso peptide has a carboxyl group; an amino group in the structure of the chitosan is connected to the carboxyl group of the lasso peptide via an amide bond.

2. The chitosan nanoparticles according to claim 1, wherein the lasso peptide includes more than one of MccJ25, MccY, Klebsidin, Citrocin, Lihuanodin, Synthetase B1, Astexin 1, Koveensin, MccS, and MccC7.

3. The chitosan nanoparticles according to claim 1, wherein the molar ratio of the chitosan to the lasso peptide is 5:(3-4).

4. The chitosan nanoparticles according to claim 1, wherein the chitosan nanoparticles further comprise arginine, and an amino group in the structure of the chitosan is connected to a carboxyl group of arginine via an amide bond.

5. A product comprising the chitosan nanoparticles according to claim 1, wherein the product has antibacterial effect against bacteria.

6. The product according to claim 5, wherein the bacteria include Salmonella, Serratia, and Klebsiella.

7. The product according to claim 5, wherein the product includes a feed, a preservative, a disinfectant, and a drug for external wound infection.

8. A method for preparing the chitosan nanoparticles according to claim 1, comprising the steps of:
1) mixing a 1-ethyl-(3-dimethylaminopropyl) carbodiimide solution with an N-hydroxysuccinimide solution to obtain an EDCN mixed solution;
2) dissolving chitosan in an acetylation solution and mixing evenly to obtain a chitosan acetylation solution; and dissolving a lasso peptide in an acetylation solution and mixing evenly to obtain a lasso peptide acetylation solution;
3) adding the EDCN mixed solution in step 1) to the chitosan acetylation solution and lasso peptide acetylation solution in step 2), respectively, and mixing evenly to obtain a chitosan-EDCN mixed solution and a lasso peptide-EDCN mixed solution; and
4) mixing the chitosan-EDCN mixed solution in step 3) with the lasso peptide-EDCN mixed solution in order and completing an esterification reaction under full stirring to prepare the chitosan nanoparticles.

9. The method according to claim 8, wherein the acetylation solution includes at least one of a sodium acetate solution, an acetyl chloride solution, an acetic anhydride solution, and a glacial acetic acid solution.

10. A method for preparing the chitosan nanoparticles according to claim 4, comprising the steps of:
1) mixing a 1-ethyl-(3-dimethylaminopropyl) carbodiimide solution with an N-hydroxysuccinimide solution to obtain an EDCN mixed solution;
2) dissolving chitosan in an acetylation solution and mixing evenly to obtain a chitosan acetylation solution; dissolving a lasso peptide in an acetylation solution and mixing evenly to obtain a lasso peptide acetylation solution; and dissolving arginine in an acetylation solution and mixing evenly to obtain an arginine acetylation solution;
3) adding the EDCN mixed solution in step 1) to the chitosan acetylation solution, the lasso peptide acetylation solution, and the arginine acetylation solution in step 2), respectively, and mixing evenly to obtain a chitosan-EDCN mixed solution, a lasso peptide-EDCN mixed solution, and an arginine-EDCN mixed solution; and
4) mixing the chitosan-EDCN mixed solution in step 3) with the lasso peptide-EDCN mixed solution and the arginine-EDCN mixed solution in order and completing an esterification reaction under full stirring to prepare the chitosan nanoparticles.

11. The method according to claim 10, wherein the acetylation solution includes at least one of a sodium acetate solution, an acetyl chloride solution, an acetic anhydride solution, and a glacial acetic acid solution.
